(19) United States
(12) Patent Application Publication
(10) Pub. No.: US 2024/0041727 A1
(43) Pub. Date: Feb. 8, 2024

(54) GLYCOSYLATED BACTERIORUBERINS AND INDUSTRIAL APPLICATIONS THEREOF

(71) Applicants: Jean-Noel THOREL, Paris (FR); Miroslav RADMAN, Le Kremlin Bicetre (FR); NAOS INSTITUTE OF LIFE SCIENCE, Aix En Provence (FR)

(72) Inventors: Jean-Noel THOREL, Paris (FR); Miroslav RADMAN, Le Kremlin Bicetre (FR)

(21) Appl. No.: 18/257,526

(22) PCT Filed: Dec. 16, 2021
(86) PCT No.: PCT/EP2021/086269

§ 371 Date: Jun. 14, 2023

(30) Foreign Application Priority Data
Dec. 16, 2020 (FR) 2013389

(51) Int. Cl. A61K 8/34; A61Q 19/08

(52) U.S. Cl. CPC A61K 8/345; A61Q 19/08; A61K 2800/85

(57) ABSTRACT

The invention concerns glycosylated bacterioruberins isolated from monoglycosylated bacterioruberins, diglycosylated bacterioruberins, triglycosylated bacterioruberins, tetraglycosylated bacterioruberins, pentaglycosylated bacterioruberins, hexaglycosylated bacterioruberins, heptaglycosylated bacterioruberins, octaglycosylated bacterioruberins, nonaglycosylated bacterioruberins, decaglycosylated bacterioruberins, undecaglycosylated bacterioruberins, and dodecaglycosylated bacterioruberins. In particular, the present invention concerns the purification or synthesis of glycosylated forms of the carotenoid bacterioruberin, as well as its applications, in particular in the fields of pharmaceuticals, dermocosmetics and nutraceuticals and biotechnology.

### FIELD

The present invention relates to glycosylated bacterioruberins. In particular, the present invention relates to the purification or synthesis of glycosylated forms of the carotenoid bacterioruberin, as well as to its applications, in particular in the fields of pharmaceuticals, dermocosmetics and nutraceuticals and biotechnology.

### PRIOR ART

Carotenoids are highly conjugated linear isoprenoid compounds which are responsible for the majority of the yellow, orange and red pigmentation observed in organisms on Earth (Armstrong, 1997). Although about a thousand different carotenoids have been identified in nature and they have very varied structural characteristics, all known carotenoids share a conjugated linear lipophilic backbone, obtained by way of highly conserved biosynthetic pathways (Britton, 2004). Carotenoids are synthesized from the linear condensation of isoprene units derived from primary metabolism (Armstrong, 1994). Covalent modifications at each end of the chain give rise to the observed structural diversity of known carotenoids (Armstrong 1997). Desaturation of the chains generates the chromophore which is characteristic of carotenoids, which results in a region of readily excitable delocalized electrons; these properties are the basis of two fundamental characteristics which are common to all carotenoids, namely their photochemical properties and their antioxidant action (Britton, 1995).

Carotenoid biosynthesis occurs in all living things, with the exception of animals in which carotenoids are introduced through diet (Britton, 1995).

The term “carotenoid” includes the molecules of the carotene and xanthophyll families.

Among the carotenoids with the greatest antioxidant potential, mention should be made of bacterioruberins, which are tetrahydroxylated carotenes containing 50 carbon atoms. Bacterioruberins and their derivatives are found in extremophilic bacteria, including haloarchaea and certain psychrophilic actinobacteria; in these microorganisms, they play an important role in the protection of DNA and membranes against solar radiation as well as the thermal and osmotic environmental stresses to which these organisms are permanently exposed (Mandelli et al., 2012).

In addition to the native forms of carotenoids, several organisms possess glycosylated derivatives, i.e. in which the ends of the hydrophobic chains are substituted with sugar residues. In most species, these glycosylated forms represent a minor fraction of the carotenoids and, as a result, have been the subject of few scientific studies. The actual function of these glycosylated forms of carotenoids has not been definitively elucidated. Studies have shown that in this group of photosynthetic bacteria, the glycosylated carotenoids are localized on the thylacoid membrane and the sugar residues present at the ends of the isoprenoid chain are required in order to ensure correct stacking of the chloroplast membrane (Mohamed et al., 2005). In non-photosynthetic organisms, the presence of sugars at the ends may provide anchors or attachment points for other molecules on the lipid membranes.

Despite the gaps in the understanding of the physiological functions of glycosylated carotenoids, it is obvious that in view of their physicochemical properties and in particular their amphiphilic character, as well as their antioxidant potential, these molecules can find major applications in several fields, in particular in the cosmetics, foodstuffs, nutraceuticals and pharmaceuticals industries.

It is known that the psychrophilic actinobacterium Arthrobacter agilis is capable of synthesizing glycosylated carotenoids, in particular glycosylated bacterioruberins. A strain of this species isolated from Antarctic ice has been studied in detail (Fong et al. 2001). It has been shown that the synthesis of carotenoids in this strain is induced in response to increased salinity of the culture medium and to low temperatures, indirectly implicating carotenoids and their glycosylated forms in the stabilization of bacterial membranes in the presence of an osmotic or thermal stress. Fong et al. also determined, by HPLC/UV analysis, that A. agilis produces several isomers of unglycosylated bacterioruberins as well as mono-, di-, and tetra-glycosylated forms of the same carotenoids; however, the individual molecules could not be further characterized because they could not be separated individually. That study therefore did not make it possible to determine the characteristics and properties of glycosylated bacterioruberins.

### SUMMARY

The aim of the present invention is to solve the technical problem consisting of providing glycosylated bacterioruberins, in particular in the isolated or purified form.

The aim of the present invention is to solve the technical problem consisting of providing compositions comprising one or more glycosylated bacterioruberins, in particular in the isolated or purified form.

The aim of the present invention is to solve the technical problem consisting of providing a method for separating and purifying at least one glycosylated bacterioruberin.

The aim of the present invention is to solve the technical problem consisting of providing an in vitro method for stabilizing proteins.

### DESCRIPTION OF THE INVENTION

Surprisingly, the Applicant has developed a method making it possible to efficiently isolate the glycosylated forms of bacterioruberin. The isolated glycosylated bacterioruberins, as well as their mixtures, have particular and novel biochemical properties which lend themselves to their industrial exploitation in several fields.

Thus, the present invention concerns a glycosylated bacterioruberin, characterized in that it is isolated and selected from monoglycosylated bacterioruberins, diglycosylated bacterioruberins, triglycosylated bacterioruberins, tetraglycosylated bacterioruberins, pentaglycosylated bacterioruberins, hexaglycosylated bacterioruberins, heptaglycosylated bacterioruberins, octaglycosylated bacterioruberins, nonaglycosylated bacterioruberins, decaglycosylated bacterioruberins, undecaglycosylated bacterioruberins and dodecaglycosylated bacterioruberins.

The present invention concerns a glycosylated bacterioruberin selected from monoglycosylated bacterioruberins, diglycosylated bacterioruberins, triglycosylated bacterioruberins and tetraglycosylated bacterioruberins.

According to one embodiment, the glycosylated bacterioruberin is separated and purified from an extract of extremophilic bacterium, preferably a bacterium from the Micrococcaceae family, advantageously Arthrobacter agilis.

In accordance with one embodiment, the glycosylated bacterioruberin is separated and purified from an extract of A. agilis carotenoids.

According to one embodiment, the glycosylated bacterioruberin has a degree of purity of at least 70%, or even 80%, preferably 90%, even more advantageously 95%, or even 99% by weight.

Advantageously, the extract is a total extract. A total extract is typically an extract from the entire bacterium in question.

In accordance with one embodiment, one or more glycosylated bacterioruberins isolated in accordance with the invention are synthesized by biotechnological synthesis or by chemical synthesis.

The term “bacterioruberin” in particular means (all-E)-(25,2′S)-bacterioruberin (CAS No 32719-43-0), also known as “α-bacterioruberin”.

“α-bacterioruberin” has the following structure:

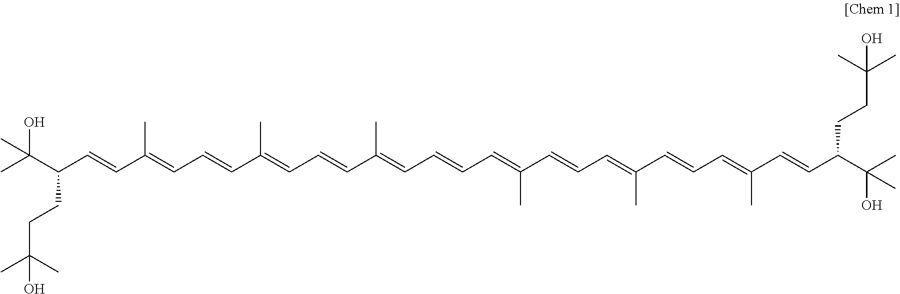 [SMILES: CC(/C=C/C=C(C)/C=C/C=C(C)/C=C/[C@H](CCC(C)(C)O)C(C)(C)O)=C\C=C\C=C(C)\C=C\C=C(C)\C=C\C=C(C)\C=C\[C@H](CCC(C)(C)O)C(C)(C)O]

α-bacterioruberin comprises 4 terminal hydroxyl groups, each of which is capable of being substituted by ether bonding with a sugar-type group, or even one or more covalently bonded sugars. The term “glycosylated form of bacterioruberin” or “glycosylated bacterioruberin” means a bacterioruberin in which at least one hydroxyl group has been substituted with one or more, for example two or three, sugar residues by means of an ether bond between the backbone of the bacterioruberin and the sugar.

An “isolated glycosylated bacterioruberin” in accordance with the invention is obtained by biotechnological synthesis, by chemical synthesis, or by purification of glycosylated bacterioruberin naturally contained in a natural bacterium.

As an example, a “glycosylated bacterioruberin” in accordance with the invention corresponds to the following structure:

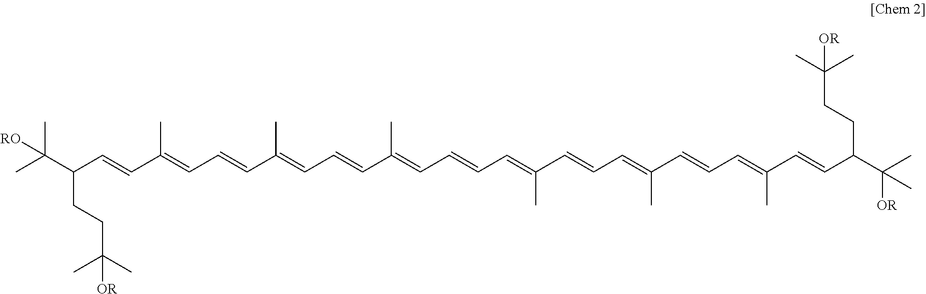

in which R is independently selected from a hydrogen atom, one or more, for example two or even three, sugar residues, and wherein in at least one occurrence, R represents one or more, for example two or even three, sugar residues.

In a preferred embodiment, the sugar is a hexose or a deoxyhexose selected from the group constituted by allose, altrose, glucose, mannose, gulose, idose, galactose, fucose, fructose and fucose.

The present invention also concerns a composition comprising at least one glycosylated bacterioruberin, said composition comprising essentially no non-glycosylated form of bacterioruberin, and said composition comprising one or more excipients acceptable to a human being.

In accordance with one embodiment, said composition comprises at least one isolated glycosylated bacterioruberin as defined in accordance with the present invention.

In accordance with one embodiment, said composition comprises a mixture of monoglycosylated bacterioruberins, diglycosylated bacterioruberins, triglycosylated bacterioruberins and tetraglycosylated bacterioruberins.

In accordance with one embodiment, said composition comprises a mixture of glycosylated bacterioruberins essentially constituted by monoglycosylated bacterioruberins and diglycosylated bacterioruberins, said mixture preferably comprising 20 to 80% by weight of monoglycosylated bacterioruberins and 20 to 80% by weight of diglycosylated bacterioruberins with respect to the total weight of the mixture of glycosylated bacterioruberins.

The present invention also concerns any one of the mixtures of two or more isolated glycosylated bacterioruberins selected from monoglycosylated bacterioruberins, diglycosylated bacterioruberins, trig lycosylated bacterioruberins, tetraglycosylated bacterioruberins, pentaglycosylated bacterioruberins, hexaglycosylated bacterioruberins, heptaglycosylated bacterioruberins, octaglycosylated bacterioruberins, nonaglycosylated bacterioruberins, decaglycosylated bacterioruberins, undecaglycosylated bacterioruberins and dodecaglycosylated bacterioruberins essentially comprising no non-glycosylated form of bacterioruberin.

Advantageously, said mixture essentially consists of a mixture of monoglycosylated bacterioruberins, diglycosylated bacterioruberins and tetraglycosylated bacterioruberins; and preferably a mixture of monoglycosylated bacterioruberins and diglycosylated bacterioruberins.

The invention also concerns a method for separating and purifying at least one glycosylated bacterioruberin defined in accordance with the invention from an extract of carotenoids containing them, comprising the following steps:
(i) dissolving said extract in a solvent, and preferably in tetrahydrofuran;
ii) separating fractions containing one or more glycosylated bacterioruberins by adsorption chromatography on a silica-type stationary phase column using an eluent, and preferably using a dichloromethane/methanol mixture.

In a preferred embodiment, the weight ratio between dichloromethane and methanol is between 20/1 and 1/20, advantageously between 10/1 and 3/7.

The fractions obtained by this purification process may be further analysed and characterized using the qualitative and quantitative methods known to the person skilled in the art such as, by way of example, thin layer chromatography, HPLC/UV, HPLC/DAD and mass spectrometry.

Advantageously, the one or more glycosylated bacterioruberin(s) is(are) separated and purified from a total carotenoid extract containing the glycosylated bacterioruberins.

Advantageously, the total extract of carotenoids containing the glycosylated bacterioruberins is a bacterial extract, preferably of Actinobacteria, even more advantageously of the Micrococcaceae family. Advantageously, the species are Micrococcus roseus and Arthrobacter agilis.

Preferably, the strains of Arthrobacter agilis used as sources of the glycosylated bacterioruberins within the meaning of the invention are the strain MB813 (described in Fong et al. 2001) and/or SB5 (described in patent application WO 2014/167247). This species is also known as Micrococcus agilis. The methods for obtaining extracts of total carotenoids of these bacterial species are known to the person skilled in the art and have been described, for example, in Strand et al. 1997, Fong et al. 2001 as well as patent application WO 2014/167247.

In a preferred embodiment, the total extract of carotenoids containing the glycosylated bacterioruberins in accordance with the invention corresponds to the extract contained in the MIRORUBERINE starting material marketed by GREENTECH and corresponding to the INCI name Micrococcus lysate. Alternatively, the glycosylated bacterioruberins in accordance with the invention may be obtained by biotechnology, or by chemical synthesis, for example by means of a controlled glycosylation of the native forms of bacterioruberins, typically of α-bacterioruberins, for example starting from the α-bacterioruberin. This glycosylation may be obtained chemically or by biotechnology, preferably by biotechnology, using one or more suitable glycosyltransferases. By way of example, the starting material HALORUBINE marketed by HALOTEK GmbH may be used as a source of α-bacterioruberin in the synthesis of glycosylated bacterioruberins within the meaning of the invention.

The glycosylated bacterioruberins in accordance with the invention, as well as the compositions containing them, have exhibited a high antioxidant activity; these molecules are capable of protecting the proteins from carbonylation and can contribute to their stabilization. Consequently, glycosylated bacterioruberins are suitable for various applications in the fields of pharmaceuticals, dermocosmetics, nutraceuticals and biotechnology.

Thus, the present invention concerns an isolated glycosylated bacterioruberin as defined in accordance with the invention, and in particular selected from monoglycosylated bacterioruberins, diglycosylated bacterioruberins, triglycosylated bacterioruberins, tetraglycosylated bacterioruberins, pentaglycosylated bacterioruberins, hexaglycosylated bacterioruberins, heptaglycosylated bacterioruberins, octaglycosylated bacterioruberins, nonaglycosylated bacterioruberins, decaglycosylated bacterioruberins, undecaglycosylated bacterioruberins, and dodecaglycosylated bacterioruberins for its use as a medicament.

The present invention further concerns an isolated glycosylated bacterioruberin selected from monoglycosylated bacterioruberins, diglycosylated bacterioruberins, triglycosylated bacterioruberins and tetraglycosylated bacterioruberins for use as a medicament.

The present invention also concerns a composition in accordance with the invention for its use as medicine.

Advantageously, said composition comprises a mixture of monoglycosylated bacterioruberins, diglycosylated bacterioruberins, triglycosylated bacterioruberins and tetraglycosylated bacterioruberins, preferably a mixture of glycosylated bacterioruberins essentially constituted by monoglycosylated bacterioruberins and diglycosylated bacterioruberins, and said mixture preferably comprising 20 to 80% by weight of monoglycosylated bacterioruberins and 20 to 80% by weight of diglycosylated bacterioruberins with respect to the total weight of the mixture of glycosylated bacterioruberins.

The invention concerns glycosylated bacterioruberins, their mixture and compositions defined in accordance with the invention, for their use in a method for the therapeutic treatment of a human being or animal. For example, it concerns the treatment of neurodegenerative diseases such as, for example, Alzheimer's disease (AD), Parkinson's disease (PD), Huntington's disease, posterior cortical atrophy or amyotrophic lateral sclerosis (ALS), as well as ocular neurodegenerative diseases selected from macular degeneration, retinitis pigmentosa and retinopathy.

However, glycosylated bacterioruberins may also be useful and therapeutically effective in the treatment of other pathologies presenting with a deregulation of protein aggregation such as fibrosis, for example, advantageously pulmonary fibrosis, and diabetes.

The pharmaceutical compositions in accordance with the invention are generally in dosage form. Thus, a composition in accordance with the invention may be in the form of a tablet, a sugar-coated tablet, a capsule, a suppository, an injectable or oral solution, or in fact a drop, and it is capable of being administered orally, oromucosally, rectally, vaginally, parenterally, intramuscularly or opthalmically.

Among the pharmaceutical compositions in accordance with the invention, more particular mention will be made of those which are suitable for oral, oromucosal, parenteral (intravenous, intramuscular or subcutaneous), per- or transcutaneous, intravaginal, rectal, nasal, perlingual, buccal, ocular or respiratory administration.

The pharmaceutical compositions in accordance with the invention for parenteral injections in particular include aqueous and non-aqueous sterile solutions, dispersions, suspensions or emulsions as well as sterile powders for reconstituting injectable solutions or dispersions.

For solid oral administration, the pharmaceutical compositions in accordance with the invention in particular include simple or sugar-coated tablets, sublingual tablets, sachets, capsules or granules, and for oral, nasal, buccal or ocular liquid administrations, in particular include emulsions, solutions, suspensions, drops, syrups and aerosols.

The pharmaceutical compositions for rectal or vaginal administration are preferably suppositories or ovules, and those for per- or transcutaneous administration in particular include powders, aerosols, creams, ointments, gels and patches.

The aforementioned pharmaceutical compositions illustrate the invention but do not limit it in any way.

Examples of excipients or vehicles which are inert, non-toxic, acceptable to a human being or pharmaceutically acceptable that may be cited by way of indication and without implying any limitation are diluents, solvents, preservatives, wetting agents, emulsifiers, dispersing agents, binders, blowing agents, disintegrating agents, retardants, lubricants, absorbents, suspending agents, dyes, flavourings, etc.

The useful dosage varies as a function of the age and weight of the patient, the mode of administration, the pharmaceutical composition used, and the nature and the severity of the condition. By way of example, the composition in accordance with the invention may be administered once a month, once a week or daily and it may contain from 1 mg to 1 g of glycosylated bacterioruberins or any of their mixtures.

The glycosylated bacterioruberins in accordance with the invention are suitable for their use in food and nutraceutical supplements. Thus, in accordance with one embodiment, a composition in accordance with the invention is a food supplement.

Methods for formulating food supplements are known to the person skilled in the art. Advantageously, these food supplements are in the form of a tablet or capsule. By way of example, each dose may contain from 1 mg to 1 g of glycosylated bacterioruberins and/or any of their mixtures.

In a tablet, microcrystalline cellulose is used as a bulking agent, for example. It is used in a quantity of between 10 and 30% by weight with respect to the total weight of the food supplement, more advantageously approximately 20% by weight.

Dicalcium phosphate and tricalcium phosphate are used as compression agents for the preparation of tablets. 10% to 30% by weight of dicalcium phosphate with respect to the total weight of the food supplement is used, more advantageously approximately 15% by weight. A quantity of 2.5% to 7.5% by weight of tricalcium phosphate with respect to the total weight of the food supplement is used, and more advantageously approximately 5% by weight.

Hydrated silica, magnesium stearate and colloidal silica may advantageously be used as thinners in the food supplement in the form of tablets or capsules. They are introduced in a quantity of approximately 2% by weight, 1% by weight and 0.6% by weight with respect to the total weight of the food supplement, respectively.

Other adjuvants such as flavourings (natural or chemical, fruit or other flavourings) or pigments are advantageously incorporated into the food supplement preparation.

When the food supplement is in the form of a soft capsule or a capsule, the envelope of these soft capsules or these capsules may in particular contain animal gelatine such as fish gelatine, glycerine, or a material of plant origin such as a cellulose or starch derivative, or a plant protein. In a preferred embodiment, one or more glycosylated bacterioruberins in accordance with the invention incorporated into the capsules may be dissolved in a fatty substance, advantageously caprylic and/or capric triglyceride and preferably stabilized with tocopherol.

Glycosylated bacterioruberins, as well as compositions thereof, are suitable for use in biotechnological applications. By way of example, these molecules may be used to stabilize proteins, for example enzymes, and to prevent or slow down their degradation, inactivation, aggregation, etc.

Thus, the present invention also concerns an in vitro method for stabilizing proteins, consisting of incubating or storing the proteins to be stabilized in contact with one or more glycosylated bacterioruberins as defined in the invention or with a composition as defined in accordance with the invention.

The properties of glycosylated bacterioruberins, and in particular their protein-protecting properties and their amphiphilic character, lend themselves to the exploitation of these molecules in the field of dermocosmetics. Thus, in a particular embodiment of the invention, a composition in accordance with the invention is a cosmetic composition, advantageously suitable for topical application.

Advantageously, said composition comprises at least one glycosylated bacterioruberin selected from monoglycosylated bacterioruberins, diglycosylated bacterioruberins, triglycosylated bacterioruberins, tetraglycosylated bacterioruberins, pentaglycosylated bacterioruberins, hexaglycosylated bacterioruberins, heptaglycosylated bacterioruberins, octaglycosylated bacterioruberins, nonaglycosylated bacterioruberins, decaglycosylated bacterioruberins, undecaglycosylated bacterioruberins and dodecaglycosylated bacterioruberins. Preferably, said composition comprises a mixture of monoglycosylated bacterioruberins, diglycosylated bacterioruberins and tetraglycosylated bacterioruberins; preferably a mixture of glycosylated bacterioruberins essentially constituted by monoglycosylated bacterioruberins and diglycosylated bacterioruberins, and said mixture preferably comprising 20 to 80% by weight of monoglycosylated bacterioruberins and 20 to 80% by weight of diglycosylated bacterioruberins with respect to the total weight of the mixture of glycosylated bacterioruberins.

In a preferred embodiment, the glycosylated bacterioruberins in accordance with the invention represent from 0.0001% to 0.5%, advantageously 0.001% to 0.01% by weight with respect to the total weight of the cosmetic composition.

In accordance with an alternative embodiment and advantageously, the composition in accordance with the invention additionally comprises at least one UV filter.

In the context of the invention, the term “UV filter” encompasses organic or inorganic compounds which are capable of filtering UV-A, UV-B and/or UV-C.

In accordance with the invention, these may also be inorganic filters instead of chemical or organic filters.

The compositions in accordance with the invention may contain one or more broad spectrum UV filters, i.e. compounds or mixtures which absorb UV-A, UV-B, UV-C and possibly visible light.

Examples of broad spectrum organic filters which may be used in the context of the invention include filters corresponding to the following INCI names: tris biphenyl triazine, bis ethylhexyloxyphenol methoxyphenyl triazine, methylene bis-benzotriazolyl tetramethylbutylphenol. By way of example, they are marketed by BASF under the names TINOSORB S®/TINOSORB AQUA®, TINOSORB A2B®, TINOSORB M®, respectively. Another example of a broad-spectrum filter suitable for the composition of the invention corresponds to the INCI name diethylhexyl butamido triazone, for example marketed by SIGMA 3V under the name UVASORB HEB®.

Thus, and in a particular embodiment, the composition in accordance with the invention comprises at least one filter selected from the following group of compounds identified by their INCI name: tris biphenyl triazine, bis ethylhexyloxyphenol methoxyphenyl triazine, and methylene bis-benzotriazolyl tetramethylbutylphenol, diethylhexyl butamido triazone, or mixtures thereof.

Advantageously, the composition comprises the filter bis-ethylhexyloxyphenol methoxyphenyl triazine.

In accordance with another embodiment, instead of or in addition to broad spectrum filter(s), the composition contains at least one UV-A and/or UV-B filter, organic and/or inorganic, which may be in the aqueous phase (lipophilic) and/or oily phase (liposoluble).

Thus, and by way of example, the composition in accordance with the invention may contain liposoluble UV-B filters which are capable of contributing to stabilizing or dissolving broad spectrum filters, or in fact to be reciprocally stabilized and for this reason, to increase the sun protection factor (SPF).

Advantageously, filters of this type correspond to the following INCI names: homosalate, octocrylene, ethylhexyl salicylate, ethylhexyl triazone.

In a preferred embodiment, the composition of the invention comprises ethylhexyl triazone, marketed by BASF under the name UVINUL T150®.

In another embodiment, the liposoluble UV-B filter is α-(trimethylsilyl)-ω-(trimethylsilyloxy)poly[oxy(dimethyl)silylene]-co-[oxy(methyl)(2-{4-[2,2-bis(ethoxycarbonyl)vinyl]phenoxy}-1-methylene ethyl)silylene]-co-[oxy(methyl)(2-(4-[2,2-bis(ethoxycarbonyl)vinyl]phenoxy)prop-1-enyl)silylene], a silicone polymer which is capable of filtering in the UV-B range. This filter corresponds, for example, to the cosmetic starting material Parsol SLX®, marketed by DSM under the INCI name polysilicone-15.

In a preferred embodiment, the composition in accordance with the invention comprises at least one UV-B filter selected from the following group of compounds identified by their INCI name: homosalate, ethylhexyl salicylate, ethylhexyl triazone, polysilicone-15, or mixtures thereof.

In a particular embodiment, the composition is free from the following filters: 4-methylbenzylidene camphor, benzophenone-2, benzophenone-3, ethylhexyl methoxycinnamate, octocrylene.

In an advantageous embodiment, the composition in accordance with the invention comprises at least one UV-A filter in order to ensure complete screening of the harmful portion of the solar spectrum.

Advantageous UVA screening agents within the meaning of the present invention are butyl methoxydibenzoylmethane (INCI) and diethylamino hydroxybenzoyl hexyl benzoate (INCI), respectively corresponding to the starting materials Parsol 1789® marketed by DSM and UVINUL® A+ marketed by BASF.

In a particular embodiment, the UV-A screening agent is bis(diethylaminohydroxybenzoyl benzoyl) piperazine (INCI) (CAS number 919803-06-8), corresponding, for example, to the starting material C1332® marketed by BASF.

Thus, and in a preferred embodiment, the composition in accordance with the invention comprises at least one filter selected from the following group of compounds identified by their INCI names: butyl methoxydibenzoylmethane, diethylamino hydroxybenzoyl hexyl benzoate, bis-(diethylaminohydroxybenzoyl benzoyl) piperazine, or mixtures thereof.

Other UV filters which are advantageous in the context of the present invention are hydrosoluble filters such as, for example:
the filter corresponding to the INCI name disodium phenyl dibenzimidazole tetrasulfonate, in particular available under the name Neo Heliopan® AP (Symrise);
the filter corresponding to the INCI name phenylbenzimidazole sulfonic acid, in particular available under the name Neo Heliopan® hydro (Symrise), preferably in combination with a basic amino acid, advantageously arginine. In practice, the basic amino acid represents between 0.5% and 2% by weight of the composition, preferably between 1% and 1.5% by weight of the composition.

In a preferred embodiment, the composition in accordance with the invention comprises at least one hydrosoluble filter selected from the following group of compounds identified by their INCI names: disodium phenyl dibenzimidazole tetrasulfonate, phenylbenzimidazole sulfonic acid, or mixtures thereof.

Advantageously, the inorganic mineral filters, or mineral sunscreens, are metallic oxides and/or other compounds which are difficult to dissolve or are insoluble in water, in particular oxides of titanium (TiO₂), zinc (ZnO) advantageously doped with iron, iron (Fe₂O₃), zirconium (ZrO₂), silicon (SiO₂), manganese (for example MnO), aluminium (Al₂O₃), or cerium (Ce₂O₃), bismuth (Bi₂O₃).

In accordance with a particular embodiment, the inorganic mineral sunscreens may be used in the form of an oily or aqueous pre-dispersion which is commercially available. These pre-dispersions may advantageously be supplemented with dispersion aids and/or solubilization aids.

The inorganic mineral filters may also be surface-treated or encapsulated, in order to provide them with a hydrophilic, amphiphilic or hydrophobic character. This surface treatment may consist of providing the mineral sunscreens with a thin inorganic and/or organic, hydrophilic and/or hydrophobic film.

In a preferred embodiment, the composition in accordance with the invention comprises at least one mineral sunscreen selected from the following group of compounds identified by their INCI names: zinc oxide, titanium dioxide, or mixtures thereof.

The cited lists of UV filters which may be used in the context of the present invention are clearly given by way of non-limiting indication.

Advantageously, the UV filters as described above which are present in the composition in accordance with the invention represent between 0.1% and 30% of the weight of the composition, advantageously between 0.5% and 20%, even more advantageously between 1% and 15%.

In accordance with a particular embodiment, the composition in accordance with the invention has a sun protection factor (SPF) which is greater than or equal to 10, preferably greater than or equal to 20, advantageously greater than or equal to 30, even more advantageously greater than or equal to 50.

In accordance with a preferred embodiment, the composition in accordance with the invention has a ratio of UV-A/UV-B protection which is greater than or equal to ⅓.

The sunscreen composition in accordance with the invention comprises at least one sunscreen solubilizer selected from the following group of compounds identified by their INCI name: caprylyl caprylate/caprate, dibutyl adipate, dicaprylyl carbonate, diisopropyl sebacate, dicaprylyl ether, cococaprylate, C12-15 alkyl benzoate, propylheptyl caprylate and butylene glycol dicaprylate/dicaprate. These solubilizers are available on the market from several suppliers. By way of example, the following starting materials may be used in the composition of the invention:
Several starting materials from the CETIOL® range marketed by BASF, in particular CETIOL® RLF, CETIOL® B, CETIOL® CC, CETIOL® O, CETIOL® C5, CETIOL® AB, CETIOL® SENSOFT, respectively corresponding to the INCI names caprylyl caprylate/caprate, dibutyl adipate, dicaprylyl carbonate, dicaprylyl ether, cococaprylate, C12-15 alkyl benzoate, propylheptyl caprylate;
DUB DIS marketed by STEARINE DUBOIS, corresponding to the INCI name diisopropyl sebacate;
MIGLYOL® 8810 marketed by 101 Oleo GmbH, corresponding to the INCI name butylene glycol dicaprylate/dicaprate.

In a preferred embodiment, the composition in accordance with the invention comprises at least four solubilizers selected from the following group of compounds identified by their INCI name: caprylyl caprylate/caprate, dibutyl adipate, dicaprylyl carbonate, diisopropyl sebacate, dicaprylyl ether, cococaprylate, C12-15 alkyl benzoate, propylheptyl caprylate and butylene glycol dicaprylate/dicaprate.

In a particular implementation, the composition in accordance with the invention comprises solubilizers corresponding to the INCI names dibutyl adipate, dicaprylyl carbonate and diisopropyl sebacate. In a preferred embodiment, the composition in accordance with the invention furthermore comprises at least one other solubilizer selected from the following group of compounds identified by their INCI names: propylheptyl caprylate, dicaprylyl ether, coco-caprylate, C12-15 alkyl benzoate, caprylyl caprylate/caprate. Advantageously, it is propylheptyl caprylate.

In accordance with a preferred embodiment, the solubilizers as defined above represent between 5% and 80% of the total weight of the composition, advantageously between 10% and 70%, more advantageously between 15% and 60%.

The composition in accordance with the invention may additionally comprise a SPF “booster”, i.e. an agent which amplifies the sun protection factor, and/or a photostabilizer, i.e. an ingredient which can be used to increase the SPF or photostabilize the filters, an ingredient of this type not itself being considered to be a sun screen. Examples which may be cited are:
butyloctyl salicylate (INCI), the photostabilizer advantageously representing between 0.01% and 10% of the composition weight, even more advantageously between 0.1% and 2%. This starting material is, for example, marketed by HALLSTAR under the name Hallbrite® BHB;
benzotriazolyl dodecyl p-cresol (INCI), the photostabilizer advantageously representing between 0.01% and 10% of the composition weight, even more advantageously between 0.1% and 2%. This starting material is, for example, marketed by BASF under the name TINOGARD® TL;
pongamol (INCI), a plant molecule absorbing in the UV-A, advantageously representing between 0.5 and 2% of the composition weight, even more advantageously of the order of 1%. By way of example, the starting material Pongamia Extract marketed by GIVAUDAN may be used in the context of the present invention;
ethylhexyl methoxycrylene (INCI), photostabilizer, solubilizer and SPF “booster”, advantageously representing between 1% and 5% of the composition weight. The SolaStay® S1 starting material marketed by HALLSTAR may be used in the context of the present invention;
a styrene-acrylate copolymer (INCI: styrene/acrylate copolymer), preferably representing between 1% and 10% of the composition weight in accordance with the invention. The starting materials SunSpheres® H53 and SunSpheres® PGL Polymer, marketed by DOW CHEMICALS, may be used in the context of the present invention;
diethylhexyl syringylidene malonate (INCI), advantageously representing between 1% and 10% of the composition weight. The starting material OXYNET® ST, marketed by MERCK, may be used in the context of the present invention;
a water-dispersible polyester, corresponding to the INCI names polyester-5 (and) sodium silicoaluminate, advantageously representing between 1% and 10% by weight of the composition, in particular EASTMANN AQ™38s polymer marketed by SAFIC-ALCAN;
an acrylate copolymer with a glass transition temperature of −5° C. to −15° C., as measured by differential scanning calorimetry, said copolymer advantageously representing between 1% and 10% of the composition weight. As an example, a polymer corresponding to the INCI name acrylate copolymer, such as the starting material EPITEX 66 marketed by DOW CHEMICALS, may be used in the context of the present invention.

In a particular embodiment, the composition in accordance with the invention also comprises one or more substances suitable for filtering visible light, in particular blue light. By way of example, the compounds described in document EP 1 484 051 may be used to ensure the filtration of blue light.

In accordance with a preferred embodiment, the composition in accordance with the invention additionally comprises other components which can contribute to internal protection by an action which may consist of protection of DNA, a reduction in immunosuppression induced by UV radiation, a radical scavenging action or a combined effect of these actions.

The protective action of a preparation in accordance with the invention against oxidative stress or against the effect of free radicals may be further improved if it also comprises one or more antioxidants, easily selected by a person skilled in the art, for example from the following list: tocopherol and its derivatives, totarol, magnolol, honokiol, amino acids and their derivatives, peptides (D and/or L-carnosine) and their derivatives (for example anserine, hypotaurine, taurine), carotenoids, carotenes (α-carotene, β-carotene, lycopene) and their derivatives, chlorogenic acid and its derivatives, lipoic acid and its derivatives (dihydrolipoic acid), aurothioglucose, propylthiouracil and other thiols (thioredoxin, glutathione, cysteine, cystine, cystamine and their glycosyl, N-acetyl, methyl, ethyl, propyl, amyl, butyl and lauryl, palmitoyl, oleyl, γ-linoleic, cholesteryl and glyceryl esters) as well as salts thereof, dilauryl thiodipropionate, distearyl thiodipropionate, thiodipropionic acid and its derivatives, sulfoximine compounds (buthionine sulfoximine, homocysteine sulfoximine, buthionine sulfones, penta-, hexa- and heptathionine sulfoximine), chelating agents (such as α-hydroxy fatty acids, palmitic acid, phytic acid, lactoferrin), α-hydroxy acids (such as citric, lactic or malic acid), humic acid, bile acid, bile extracts, bilirubin, biliverdin, EDTA, EGTA, ethylenediamine pentasodium tetramethylene phosphonate and its derivatives, unsaturated fatty acids and their derivatives, vitamin A and its derivatives (vitamin A palmitate), coniferyl benzoate of benzoin resin, rutinic acid and its derivatives, α-glycosyl rutin, ferulic acid and its derivatives, furfurylidene glucitol, carnosine, butyl hydroxytoluene, butyl hydroxyanisole, norhydroguairetic acid, trihydroxybutyrophenone, quercetin, uric acid and its derivatives, mannose and its derivatives, zinc and its derivatives (ZnO, ZnSO₄), selenium and its derivatives (selenomethionine), stilbene and its derivatives (stilbene oxide, trans-stilbene oxide).

In a particular embodiment, the composition in accordance with the invention also contains glycyrrhetinic acid, a derivative or a salt of this acid, used as a soothing agent (anti-inflammatory agent) and representing between 0.01% and 2% by weight of the composition, preferably between 0.1% and 1%.

In accordance with another embodiment of the invention, the cosmetic and/or dermatological composition contains at least one, or even all of the following constituents exerting an in vivo biological effect on the cells of the skin, the lips, the hair and/or the mucosae subjected to UV-A and/or UV-B radiation, respectively:
a radical scavenger preserving cell structures, such as, for example, vitamin E and/or its liposoluble or hydrosoluble derivatives, in particular tocotrienol and/or tocopherol, advantageously representing between 0.001% and 10% of the total weight of the composition, even more advantageously between 0.02% and 2%, preferably of the order of 0.04%;
an agent which limits immunosuppression, such as vitamin PP, for example, advantageously representing between 0.001% and 1% of the total weight of the composition, more preferably between 0.01% and 0.3%;
a protective agent for the p53 protein such as, for example, epigallocatechin gallate (EGCG), advantageously representing between 0.001% and 0.1% of the total weight of the composition, more advantageously between 0.005% and 0.05%.

The composition in accordance with the invention may furthermore comprise peptide extracts of soya and/or wheat, such as those described in EP 2 059 230.

In practice, the peptide extracts deriving from soya or wheat grains are obtained by an enzymatic hydrolysis of said grains via peptidases, which means that peptides with a mean size of 700 Dalton can be recovered. Preferably, the soya peptide extract is the extract identified by the CAS number 68607-88-5 and the wheat peptide extract is the extract identified by the CAS number 70084-87-6. The wheat and soya extracts may correspond to the INCI names Hydrolyzed wheat protein and Hydrolyzed soy protein, respectively.

In a particular embodiment, the soya and wheat peptide extracts are used together, for example in a ratio by weight respectively comprised between 80/20 and 20/80, advantageously comprised between 70/30 and 30/70, preferably equal to 60/40.

In one advantageous embodiment, the soya and/or wheat peptide extracts are free from synthetic GHK tripeptides (glycyl-histidyl-lysine; INCI: Tripeptide-1). In practice, the peptide extracts of soya and/or wheat represent 0.01% to 20% of the total weight of the composition, advantageously 0.1% to 10%, more preferably 0.2% to 0.7%.

In an alternative embodiment, the composition in accordance with the invention comprises, in accordance with the teachings of document FR 2 865 398, the combination of at least one amino acid selected from the group constituted by ectoin, creatine, ergothioneine and/or carnosine, or their physiologically acceptable salts, and mannitol or a mannitol derivative.

Preferably, the composition in accordance with the invention comprises, in a physiologically acceptable medium, the amino acid or one of its salts, alone or as a mixture, in proportions comprised between 0.001% and 10% of the total weight of the composition, and preferably between 0.01% and 5%.

The composition in accordance with the present invention preferably comprises, in a physiologically acceptable medium, mannitol or one of its derivatives, in proportions comprised between 0.01% and 30% of the total weight of the composition, advantageously between 0.1% and 10%.

In a preferred embodiment, the composition in accordance with the invention comprises ectoin and mannitol.

In accordance with a particular embodiment, the composition in accordance with the invention contains one or more tanning or self-tanning agents. It may be a self-tanning agent which reacts with the amino acids of the skin in accordance with a Maillard reaction or via a Michael addition, or in fact a melanogenesis promoter or a pro-pigmenting compound which promotes natural tanning of the skin.

Such a substance is preferably present in the composition in a quantity ranging from 0.01% to 20% of the total weight of the composition, advantageously 0.5% to 15%, even more advantageously 1% to 8%.

The self-tanning substances may be 1,3-dihydroxyacetone (DHA), glycerolaldehyde, hydroxymethylglyoxal, γ-dialdehyde, erythrulose, 6-aldo-D-fructose, ninhydrin, 5-hydroxy-1,4-naphthoquinone (juglone), 2-hydroxy-1,4-naphthoquinone (lawsone), or a combination thereof.

The pro-pigmenting substances may be melanocyte-stimulating hormone (α-MSH), peptide analogues of α-MSH, endothelin-1 receptor agonists, μ opioid receptor agonists, AMPc-stimulating agents, tyrosinase-stimulating agents.

In a particular embodiment, the composition in accordance with the invention also comprises, as a self-tanning agent, a combination of dihydroxy methylchromonyl palmitate and/or dimethylmethoxy chromanol, as well as a lipophilic form of tyrosine.

This combination of active principles makes it possible to effectively stimulate tanning. Dihydroxy methylchromonyl palmitate (CAS number: 1387636-35-2) corresponds, for example, to the cosmetic ingredient marketed by Merck under the name RonaCare® Bronzyl. Dimethylmethoxy chromanol (CAS number: 83923-51-7) corresponds to the cosmetic ingredient marketed by LIPOTEC SA under the name lipochromone-6.

In a particular embodiment, the dihydroxy methylchromonyl palmitate or dimethylmethoxy chromanol is present in the composition in accordance with the invention in an amount of 0.01% to 10% of the total weight of the composition, advantageously 0.05% to 10%, yet more advantageously 0.1% to 5%, and more particularly 0.1% to 0.5%.

The lipophilic form of tyrosine, within the meaning of the invention, is an ingredient based on tyrosine and having a lipophilic character which is more pronounced than that of tyrosine. The lipophilic form of tyrosine may in particular correspond to oleoyl tyrosine (CAS number: 147732-57-8), which is found, for example, in the liquid cosmetic ingredient TYR-OL, marketed by SEDERMA, and which comprises approximately 50% by weight of oleoyl tyrosine in butylene glycol (approximately 30% +approximately 20% oleic acid), or in fact in the liquid cosmetic ingredient TYR-EXCEL, marketed by SEDERMA, which comprises approximately 50% by weight oleoyl tyrosine, approximately 20% oleic acid (CAS No.: 112-80-1) and approximately 30% Luffa cylindrica oil (sponge pumpkin seed oil; CAS No.: 1242417-48-6).

In accordance with another embodiment, the lipophilic form of tyrosine corresponds to a vegetable oil which includes tyrosine in the formulation.

In a particular implementation, the vegetable oil is oleic sunflower oil, advantageously deodorized. Thus, the starting material OLEOACTIF TYROSINE BASE HELIANTHUS ANNUS marketed by OLEOS, and corresponding to the INCI names Helianthus annuus seed oil (and) tyrosine (and) glyceryl stearate, may be used in the context of the present invention.

In practice, the lipophilic form of tyrosine, such as in the cosmetic ingredients based on oleoyl-tyrosine (advantageously 50% by weight) or of tyrosine formulated in vegetable oil, represents between 0.1% and 10% of the total weight of the composition, advantageously between 1 and 3%, even more advantageously between 1% and 1.5%.

The composition in accordance with the invention may also contain active ingredients having depigmenting properties such as, for example:
lysine azeilate, or other derivatives or salts of azelaic acid
andrographolide, in particular the extract of Andrographis paniculata corresponding to the INCI name Andrographis paniculata leaf extract;
native ascorbic acid (vitamin C) or its derivatives, in particular the derivatives corresponding to the INCI names ascorbyl glucoside ethyl ascorbic acid, ascorbyl methylsilanol pectinate, Sodium ascorbyl phosphate and ascorbyl tetraisopalmitate;
arbutin or a plant extract containing it, in particular the busserola extract corresponding to the INCI name Arctostaphylos uva-ursi leaf extract;
glabridine or a plant extract containing it, in particular liquorice extracts corresponding to the INCI name Glycyrrhiza glabra root extract, Glycyrrhiza inflata root extract, Glycyrrhiza uralensis root extract;
the biomimetic peptides corresponding to the INCI names hexapeptide 2 and/or nonapeptide-1;
an aqueous extract of an alga known as Palmaria palmata, in particular the extract corresponding to the INCI name Palmaria palmata extract;
4-n-butylresorcinol;
vitamin PP, also called niacinamide or nicotinamide, and derivatives thereof;
or mixtures thereof.

The composition in accordance with the invention may also contain active ingredients having healing properties such as, for example, an antimicrobial agent selected from the active ingredients corresponding to the following INCI names: copper sulfate, zinc sulfate, sodium hyaluronate, Vitis vinifera (grape) vine extract, or mixtures thereof.

The cosmetic composition in accordance with the invention may also contain active ingredients having sebocorrecting, keratolytic, seboregulating and/or anti-acne properties, in order to make it possible to formulate sunscreen products for treating acne.

For example, the cosmetic composition in accordance with the invention may comprise an antimicrobial agent selected from the active agents corresponding to the INCI names propyl gallate, dodecyl gallate, Ginkgo biloba leaf extract, bakuchiol, dihydromyricetin, zinc gluconate, salicylic acid, or mixtures thereof.

The composition in accordance with the invention may therefore further comprise at least one ingredient selected from the following list:
at least one polyol selected from xylitol, rhamnose, sorbitol and mannitol;
an extract of the algae Laminaria ochroleuca, Blidingia minima or Laminaria saccharina;
an extract of the plant Zanthoxylum alatum;
panthenol;
vitamin E or a hydrophilic or lipophilic derivative thereof, or a salt thereof, advantageously tocotrienol or tocopherol;
salicylic acid or a derivative thereof;
a cade wood extract;
a boldo extract;
a meadowsweet extract;
glycyrrhetinic acid or a derivative or salt thereof;
an agent for limiting immunosuppression, advantageously vitamin PP;
a protective agent for the p53 protein, advantageously epigallocatechin gallate (EGCG);
an extract of karanja oil obtained from Pongamia glabra;
linear paraffins;
ATP (adenosine-5 triphosphate), Gp₄G (diguanosine tetraphosphate) or Ap₄A (diadenosine tetraphosphate),
an agent limiting the action of the iron ions involved in the formation of free radicals, advantageously EDTA;
a peptide extract of soya and/or wheat;
an amino acid selected from the group consisting of ectoin, creatine, ergothioneine, carnosine, tyrosine, decarboxycarnosine, glutamine and salts thereof;
a tanning or self-tanning agent;
a depigmenting agent;
a healing agent;
a sebocorrective, keratolytic, seboregulatory and/or anti-acne agent.

The composition in accordance with the invention may also contain adjuvants such as those usually used in the field of cosmetics, such as active ingredients, preservatives, antioxidants, complexing agents, solvents, fragrances, fillers, bactericides, electrolytes, odour absorbers, dyestuffs or lipid vesicles. The choice of these adjuvants, as well as their concentrations, must be determined in such a way that they do not modify the desired properties and the advantages of the composition of the present invention.

The composition in accordance with the invention is intended for topical application and more particularly for application to the skin, lips, hair and/or mucous membranes.

The composition in accordance with the invention may be presented in any of the galenical forms normally used in the cosmetic and dermatological fields such as, for example, but in a non-limiting manner, in the form of an optionally gelled aqueous solution, a dispersion of the lotion type, a (O/W) or conversely (W/O) emulsion, which is fluid to a greater or lesser extent, or a multiple emulsion such as, for example, a triple emulsion (W/O/W or O/W/O), or in fact in the form of a vesicular dispersion of the ionic type (liposomes) and/or non-ionic type, a two-phase composition free from emulsifiers and gelling agents, the immiscible phases of which separate during storage, a foam, a stick, an anhydrous oil, a spray or a mist.

In a preferred embodiment, the composition in accordance with the invention is a W/O emulsion. Advantageously, the W/O emulsions in accordance with the invention comprise emulsifying quality PEG-30 dipolyhydroxystearate (INCI).

In an alternative embodiment, the composition in accordance with the invention is an O/W emulsion. Advantageously, the O/W emulsions in accordance with the invention comprise an emulsifier selected from the following group of compounds identified by their INCI name: potassium cetyl phosphate, glyceryl stearate, PEG-100 stearate and C20-22 alkyl phosphate/C20-C22 alkyl alcohol, glyceryl stearate citrate, tribehenin PEG-20 esters, and mixtures thereof.

The composition in accordance with the invention may also comprise preservatives. Any preservative which is capable of being used in cosmetic or dermatological compositions may be used in the formulation of a composition in accordance with the invention.

Advantageously, the preservatives used are alkanediols, even more advantageously 1,2-alkanediols or 1,3-alkanediols, and mixtures thereof.

In a preferred embodiment, the preservative is a 1,2-diol selected from 1,2-pentanediol, 1,2-hexanediol, 1,2-heptanediol, 1,2-octanediol and 1,2-decanediol, or mixtures thereof.

In an alternative embodiment, the preservative is a 1,3-diol selected from 1,3-propanediol and 1,3-butanediol, or mixtures thereof. In practice, these alkanediols are marketed by several companies, in particular SYMRISE or MINACARE.

Preferably, the composition in accordance with the invention comprises between 0.01% and 2% by weight of the diol composition, advantageously between 0.1% and 1%, for example 0.5%.

The present invention also concerns a cosmetic, non-therapeutic treatment method, advantageously for combatting oxidative stress, and in particular cellular ageing of the skin, consisting of applying a composition in accordance with the invention to the skin of a subject in need thereof.

Advantageously, said composition comprises a mixture of monoglycosylated bacterioruberins, diglycosylated bacterioruberins, and tetraglycosylated bacterioruberins; and preferably a mixture of glycosylated bacterioruberins essentially constituted by monoglycosylated bacterioruberins and diglycosylated bacterioruberins, and said mixture preferably comprising 20 to 80% by weight of monoglycosylated bacterioruberins and 20 to 80% by weight of diglycosylated bacterioruberins with respect to the total weight of the mixture of glycosylated bacterioruberins.

Said composition, advantageously applied topically, is also suitable for the following uses:
an agent for combatting cellular aging of the skin due to all or a portion of solar radiation, typically UV radiation or visible light;
a radical scavenger or antioxidant;
as a protective agent against protein degradation;
a protective agent for proteome degradation;
a protective agent for cellular detoxification mechanisms; or
a protective agent for DNA repair systems.

The manner in which the invention may be implemented and the concomitant advantages will become more apparent from the following exemplary embodiments, which are given by way of non-limited indication and with the support of the accompanying figures.

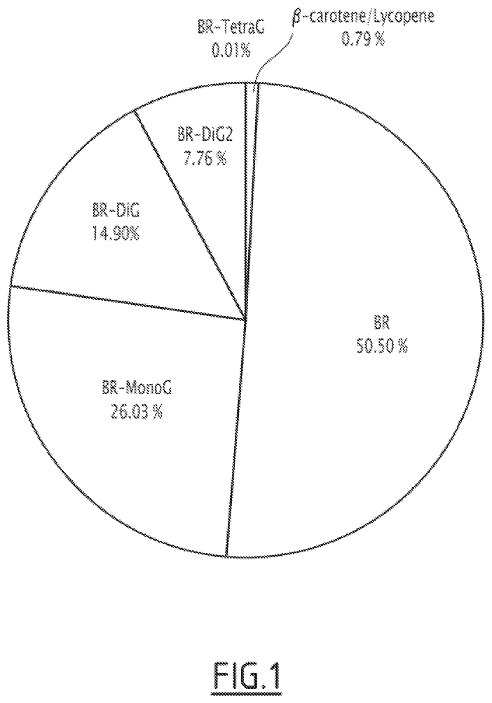

FIG. 1 shows the composition of an extract of carotenoids from the isolate SB5 of the species A. agilis.: BR=α-bacterioruberin, BR-MonoG: monoglycosylated form, BR-DiG: diglycosylated form, BR-DiG2: Another form of BR-DiG, BR-TetraG: tetraglycosylated form.

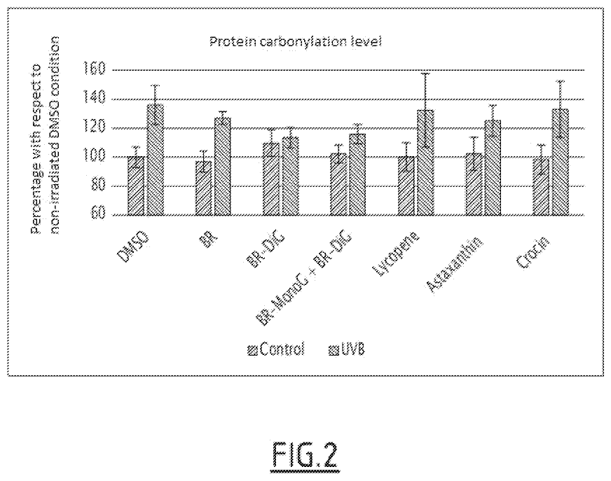

FIG. 2 shows the protection provided by several carotenoids against UV-B-induced protein carbonylation in Normal Human Keratinocytes (NHK). DMSO=dimethyl sulphoxide (solvent control); BR=α-bacterioruberin; BR-DiG=diglycosylated bacterioruberins;: BR-MonoG+BR-DiG=mixture of mono- and di-glycosylated bacterioruberins.

### EXEMPLARY EMBODIMENTS

### Example I

### Purification of the Glycosylated Bacterioruberins of an Extract of Carotenoids of the Bacterium A. agilis

### I-1 Aim of the Study

The aim of this study was to isolate and quantify the molecules contained in an extract of total carotenoids from the bacterium A. agilis.

### I-2 Materials and Methods

### I-2.1 Extract

The total extract of carotenoids from the bacterium A. agilis contained in the GREENTECH starting material known as “Miroruberine” and corresponding to the INCI name Micrococcus lysate was used in this study; it was derived from the strain SB5. This so-called “SBE” extract could be obtained by using the method described in the publication of patent application WO 2014/167247.

### I-2.2 Column and Thin-Layer Chromatography

The SBE extract was taken up in tetrahydrofuran (THF) until it had completely dissolved. A step for separating the glycosylated Bacterioruberins by silica gel chromatography was carried out in a glass column after diluting the SBE in THF.

1. Suspension of the silica gel in a DCM/methanol mixture (10/1) before being poured into the column

2. After sedimentation of the silica gel, 1 cm of sand is added before carrying out 3 washes with the DCM/methanol mixture.

3. 0.5 mL of SBE diluted in THF was deposited on the sand and allowed to stand for 5 minutes

4. 50 mL of DCM/methanol mixture (10/1) was added slowly, allowing fractions 1, 2, 3 and 4 to be collected

5. 40 mL of DCM/methanol mixture (8/2) was added slowly, allowing the collection of fractions 5 and 6

6. 40 mL of DCM/methanol mixture (5/5) was added slowly, allowing the collection of fraction 7

7. 40 mL of DCM/methanol mixture (3/7) was added slowly to allow the collection of fraction 8

8. All of the fractions were then compared by TLC (DCM/methanol (10/1)) with SBE and quantified by absorption (using the absorption maximum for each fraction).

### I-2.3 Separation of Fractions by HPLC

Equipment: Nexera XR, binary pump (Shimadzu)

Column: C18; Intersustainable Swift 5 μm 4.6×150 mm, manufacturer: GL Sciences

Mobile phases:

A: 20% H₂O in MeOH

B 20% EtOAc in MeOH

Flow: 1.5 mL/min

Injection volume: 50 μL

TABLE 1

| | A | B |
|---|---|---|
| 1 min | 100 | 0 |
| 20 min | 0 | 100 |

### I-3 Results and Discussion

In this purification, the first step for separation by column chromatography made it possible to collect the various fractions the purity of which was confirmed by TLC and by HPLC-DAD, by comparing it with the absorbance spectrum for the native extract; the quantification of the various forms was carried out by UV absorption at 500 nm.

Each of the molecules of each of the fractions collected by chromatography was identified by Maldi-TOF-TOF spectroscopy using an AUTOFLEX instrument (Brucker: method: CHCA and DHB Matrix without TFA in reflector acquisition). The distribution between the different forms was calculated by combining the results obtained with the quantifications carried out on these fractions by HPLC-DAD (FIG. 1). Fraction 1 corresponds to beta-carotene, a by-product of the synthesis of bacterioruberin, but this molecule represented only 0.79% of the extract. Fraction 4 had the same extract profile for Halobacter salinarium (Halorubin), the majority molecule of which is bacterioruberin (BR). This molecule represents approximately half of the dry extract (FIG. 1).

Two diglycosylated forms (BR-DiG1 and BR-DiG2) which migrated separately were identified. The diglycosylated forms represented >22% of the extract.

The monoglycosylated form BR-MonoG represented >26% of the extract. The tetraglycosylated form (BR-TetraG) represented only 0.01% of the extract (FIG. 1).

### Example II

### Protection Conferred by Several Carotenoids against UV-B-Induced Carbonylation in Human Keratinocytes

### II-1 Aim of the Study

The aim of this study was to compare the effect of protecting the keratinocyte proteins from UV stress for the various molecules derived from a carotenoid extract of the SB5 strain of the actinobacterium A. agilis according to Example I. In particular, the following molecules were the subject of the study:
α-bacterioruberin (BR)
the monoglycosylated form of bacterioruberin (MonoG-BR)
the two diglycosylated forms of bacterioruberin, (BR-DiG1 and BR-DiG2); these forms were combined and studied together (BR-DiG)

A known carotene, a xanthophyll and a glycosylated carotenoid were also tested:
lycopene (carotene)
astaxanthin (xanthophyll)
crocin (glycosylated carotenoid).

The total carbonylation of the proteins was measured in the presence or absence of UV-B stress.

### II-2 Materials and Methods

### II-2.1 Experimental Design

1—Test different stress doses (UV) on 12 wp (“well plate”) (aggregation):
UV-B (80; 40; 20; 15; 10 mJ/cm2)

2—Based on 1-, test 2 different stress doses (UV) on a small flask for carbonylation;

3—Test the molecules against UV stress on 54 flasks for carbonylation: 8 molecules+control, in triplicate, with and without UV;

4—Measure the carbonylation of the 108 conditions (in duplicate).

### II-2.2 Cell Treatment and Irradiation

UV-B stress: Lamp at 313 nm, dose of 80; 40; 20; 10mJ/cm² for dose selection and 15 mJ/cm² for molecule testing

### II-2.3 Test Molecules and Concentrations

BR, 100 nM
BR-DiG, 100 nM
BR-MonoG +BR-DiG 100 nM (50+50 nM)
lycopene 100 nM
astaxanthin 100 nM
crocin 100 nM.
the solvent (DMSO) represented the control.

### II-2.4 Culture and Treatment of Keratinocytes

Immortalized NHEK/SVTERT 3-5 keratinocytes were seeded with approximately one million cells/plate in 108 plates (diam: 100 mm, 56 cm²), corresponding to 36 conditions in the biological triplicates—the cells were cultured to a confluence of 90 to 95% in the keratinocyte medium (Keratinocyte-SFM from Gibco, 17005075) at 37° C. and 5% CO₂.

The cells were incubated with the test molecules for 2 hours.

After 2 h of pretreatment, the medium was removed and replaced with PBS. Half of the plates were irradiated with 15 mJ/cm² UV-B.

Following the irradiation in each plate, Keratinocyte-SFM medium was added and the cells were incubated for 24 h at 37° C., 5% CO₂.

After removing the medium from all the plates, the cells were washed with PBS and then trypsinized with trypsin-EDTA (0.25%) in HBSS (1×) in the presence of phenol red (Capricorn Scientific, TRY-3B). After collecting the plates, the cells were counted, transferred into 1.5 mL tubes and the pellets were stored at −80° C.

### II-2.5 Measurement of Protein Carbonylation

The cells were lysed by adding 100 μL of UTC lysis buffer (UTC: urea, thiourea & CHAPS) to each tube containing the cell pellet. The lysis of the cells was carried out on a microtube shaker at a temperature of 4° C. for 45 min; 600 rpm.

After that, the samples were centrifuged and the supernatant was collected; the granules were discarded.

The supernatant was transferred into a 3 kDa Amicon filter and rinsed with PBS (approximately 100 μL of protein for 400 μL of PBS). The samples were centrifuged for 40 min at +4° C. at 14000 rpm. The procedure was repeated 3 times for each sample. After the third centrifugation, the Amicon filters were placed in a new tube and centrifuged for 3 minutes at 2000 rpm; the eluates were used in the remainder of the analysis.

The protein concentration was measured by means of a Bradford protein quantification assay.

Each 15 μg sample of protein was stained with 0.15 μL of 5 mM CF647 dye. The staining was carried out overnight at +4° C., 600 rpm.

The protein samples were loaded onto 12.5% acrylamide gels. The electrophoresis was carried out at 80V until the samples were removed from the stacking gel, after which the voltage was increased to 120V until the samples reached the end of the gel.

The gels were then collected and rinsed several times in ultrapure water. Next, the gels were stained with 1× purple dye (SERVA Purple −43386.01) from a 250× concentrated form in order to examine the expression of the proteins. This step was carried out at 50 rpm on a shaker for 10 min at ambient temperature.

The images were digitized with the Amersham™ Typhoon™ Biomolecular Imager for both dyes. Fluorescence scanning was at 635 nm for CF647 and at 532 nm for SERVA violet.

Subsequently, the analysis of the scan was carried out with Life Science Research Bio-Rad's Image Lab software by selecting each channel and normalizing for background noise.

The total carbonylation of each culture was finally measured using a Western blot method (Oxy-blot).

### II-3 Results and Discussions

The overall results of the experiments are shown in FIG. 2. In this figure, the levels of carbonylation before and after UV-B stress are compared. Comparison with the DMSO solvent, which should not offer significant protection, makes it possible to determine whether a compound or mixtures of compounds protects the cellular proteins from UV-B-induced carbonylation. Surprisingly, diglycosylated bacterioruberins (BR-DiG) and the mixture of mono- and diglycosylated bacterioruberins (BR-MonoG+BR-DiG) protect more effectively than all the other carotenes and xanthophylls which were tested, including crocin, which is a glycosylated carotenoid. The protection conferred in each case is greater than that observed with α-bacterioruberin (BR).

### Example III

### O/W SPF50+Emulsion

The percentages indicated are given by weight of product with respect to the total weight of the composition in the tables below.

TABLE 2

| INCI name | % |
|---|---|
| Aqua/water/eau | 49.816 |
| Octocrylene | 9.00 |
| Dicaprylyl carbonate | 8.4973 |
| Methylene bis-benzotriazolyl | 6.00 |
| tetramethylbutylphenol [nano] | |
| Glycerin | 5.00 |
| Butyl methoxydibenzoylmethane | 4.50 |
| Dimethicone | 3.00 |
| Tribehenin PEG-20 esters | 3.00 |
| Bis-ethylhexyloxyphenol methoxyphenyl triazine | 2.50 |
| C10-18 triglycerides | 2.00 |
| Methylpropanediol | 1.40 |
| Sucrose stearate | 1.00 |
| Decyl glucoside | 0.90 |
| Hydroxyethyl acrylate/sodium acryloyldimethyl | 0.88 |
| taurate copolymer | |
| Pentylene glycol | 0.50 |
| Tocopheryl acetate | 0.50 |
| Sodium citrate | 0.20 |
| 1,2-hexanediol | 0.125 |
| Caprylyl glycol | 0.125 |
| Mannitol | 0.10 |
| Xylitol | 0.10 |
| Citric acid | 0.09 |
| Polysorbate 60 | 0.06 |
| Sorbitan isostearate | 0.06 |
| Bacterioruberin monoglycoside (in accordance | 0.50 |
| with the invention) | |
| Bacterioruberin diglycoside (in accordance | 0.50 |
| with the invention) | |
| Rhamnose | 0.05 |
| Dipropylene glycol | 0.048 |
| Xanthan gum | 0.024 |
| Ectoin | 0.01 |
| Glycyrrhiza glabra (licorice) root extract | 0.01 |
| Tocopherol | 0.0027 |
| Fructooligosaccharides | 0.001 |
| Caprylic/capric triglyceride | 0.00095 |
| Laminaria ochroleuca extract | 0.00005 |

### Example IV

### Day Cream—O/W Emulsion

TABLE 3

| INCI name | % |
|---|---|
| Aqua/water/eau | 55.788 |
| Dicaprylyl carbonate | 25.0873 |
| Glycerin | 5.00 |
| Dimethicone | 3.00 |
| Tribehenin PEG-20 esters | 3.00 |
| C10-18 triglycerides | 2.00 |
| Methylpropanediol | 1.40 |
| Sucrose stearate | 1.00 |
| Hydroxyethyl acrylate/sodium acryloyldimethyl | 0.88 |
| taurate copolymer | |
| Pentylene glycol | 0.50 |
| Tocopheryl acetate | 0.50 |
| Sodium citrate | 0.20 |
| 1,2-hexanediol | 0.125 |
| Caprylyl glycol | 0.125 |
| Mannitol | 0.10 |
| Xylitol | 0.10 |
| Citric acid | 0.09 |
| Polysorbate 60 | 0.06 |
| Sorbitan isostearate | 0.06 |
| Rhamnose | 0.05 |
| Bacterioruberin monoglycoside (in accordance | 0.04 |
| with the invention) | |
| Bacterioruberin diglycoside (in accordance | 0.03 |
| with the invention) | |
| Bacterioruberin tetraglycoside (in accordance | 0.03 |
| with the invention) | |
| Ectoin | 0.01 |
| Glycyrrhiza glabra (licorice) root extract | 0.01 |
| Tocopherol | 0.0027 |
| Fructooligosaccharides | 0.001 |
| Caprylic/capric triglyceride | 0.00095 |
| Laminaria ochroleuca extract | 0.00005 |

### Example V

### Day Cream—Serum

TABLE 4

| INCI name | % |
|---|---|
| Aqua/water/eau | 70.044252 |
| Isostearyl isostearate | 6.00 |
| Limnanthes alba (meadowfoam) seed oil | 5.9982 |
| Glycerin | 5.00 |
| Propanediol | 3.00 |
| Boron nitride | 2.00 |
| Lauroyl lysine | 2.00 |
| C10-18 triglycerides | 1.50 |
| C12-16 alcohols | 1.20 |
| Pentylene glycol | 1.00 |
| Hydrogenated lecithin | 0.40 |
| Palmitic acid | 0.40 |
| Caprylic/capric triglyceride | 0.29928 |
| 1,2-hexanediol | 0.25 |
| Caprylyl glycol | 0.25 |
| Sodium polyacrylate | 0.25 |
| Fragrance (parfum) | 0.20 |
| Xanthan gum | 0.18 |
| Sclerotium gum | 0.08 |
| Lecithin | 0.076 |
| Bacterioruberin tetraglycoside (in | 0.06 |
| accordance with the invention) | |
| Pullulan | 0.06 |
| Silica | 0.004 |

### BIBLIOGRAPHY

Armstrong G A (1994) Eubacteria show their true colors—genetics of carotenoid pigment biosynthesis from microbes to plants. J Bacteriol. 176:4795-4802.

Armstrong, G A (1997). Genetics of eubacterial carotenoid biosynthesis: A colorful tale. In: Ornston, LN., editor. Annu Rev Microbiol. USA: Annual Reviews Inc.; 629-659.

Britton G. (1995) Structure and properties of carotenoids in relation to function. FASEB J. 9:1551-1558.

Britton, G L-JSPH. (2004) Carotenoids handbook. Basel; Boston: Birkhäuser Verlag.

Fong N, Burgess M, Barrow K, Glenn D (2001) Carotenoid accumulation in the psychrotrophic bacterium Arthrobacter agilis in response to thermal and salt stress Appl Microbiol Biotechnol 56: 750-756.

Mandelli F, Miranda V S, Rodrigues E, Mercadante A Z (2012) Identification of carotenoids with high antioxidant capacity produced by extremophile microorganisms. world J Microbiol Biotechnol 28:1781-1790.

Mohamed H E, van de Meene A M L, Roberson R W, Vermaas W F J. Myxoxanthophyll is required for normal cell wall structure and thylakoid organization in the cyanobacterium, Synechocystis sp strain PCC 6803 (2005) J Bacteriol. 187:6883-6892.

Strand A, Shivaji, S, Liaaen-Jensen (1997) Bacterial carotenoids 55. C50-carotenoids 25: revised structures of carotenoids associated with membranes in psychrotrophic Micrococcus roseus. Bioch. Syst. & Eco. 25: 547-552.

1-15. (canceled)

16. An isolated glycosylated bacterioruberin, wherein the glycosylated bacterioruberin is a monoglycosylated bacterioruberin, diglycosylated bacterioruberin, triglycosylated bacterioruberin, tetraglycosylated bacterioruberin, pentaglycosylated bacterioruberin, hexaglycosylated bacterioruberin, heptaglycosylated bacterioruberin, octaglycosylated bacterioruberin, nonaglycosylated bacterioruberin, decaglycosylated bacterioruberin, undecaglycosylated bacterioruberin, or dodecaglycosylated bacterioruberin, and
wherein the glycosylated bacterioruberin has a purity of at least 70% by weight.

17. The isolated glycosylated bacterioruberin of claim 16, wherein the glycosylated bacterioruberin has a purity of at least 80%, 90%, 95%, or 99% by weight.

18. The isolated glycosylated bacterioruberin of claim 16, wherein the glycosylated bacterioruberin is a monoglycosylated bacterioruberin, diglycosylated bacterioruberin, triglycosylated bacterioruberin, or tetraglycosylated bacterioruberin.

19. The isolated glycosylated bacterioruberin of claim 16, wherein the glycosylated bacterioruberin is separated and purified from an extract of an extremophilic bacterium.

20. The isolated glycosylated bacterioruberin of claim 19, wherein the extremophilic bacterium is of the Micrococcaceae family.

21. A composition comprising at least one glycosylated bacterioruberin, wherein the composition does not comprise a non-glycosylated form of bacterioruberin, and wherein the composition comprises one or more pharmaceutically acceptable excipients.

22. The composition of claim 21, wherein the composition comprises at least one isolated glycosylated bacterioruberin, wherein the glycosylated bacterioruberin is a monoglycosylated bacterioruberin, diglycosylated bacterioruberin, triglycosylated bacterioruberin, tetraglycosylated bacterioruberin, pentaglycosylated bacterioruberin, hexaglycosylated bacterioruberin, heptaglycosylated bacterioruberin, octaglycosylated bacterioruberin, nonaglycosylated bacterioruberin, decaglycosylated bacterioruberin, undecaglycosylated bacterioruberin, or dodecaglycosylated bacterioruberin, and wherein the glycosylated bacterioruberin has a purity of at least 70% by weight.

23. The composition of claim 21, wherein the composition comprises a mixture of monoglycosylated bacterioruberins, diglycosylated bacterioruberins, triglycosylated bacterioruberins, and tetraglycosylated bacterioruberins.

24. The composition of claim 21, wherein the composition comprises a mixture of glycosylated bacterioruberins comprising monoglycosylated bacterioruberins and diglycosylated bacterioruberins, and wherein the mixture comprises 20 to 80% by weight of monoglycosylated bacterioruberins and 20 to 80% by weight of diglycosylated bacterioruberins with respect to the total weight of the mixture of glycosylated bacterioruberins.

25. The composition of claim 21, wherein the composition is formulated as a therapeutic formulation.

26. The composition of claim 21, wherein the composition is in the form of a food supplement.

27. The composition of claim 21, wherein the composition is formulated as a cosmetic composition suitable for topical application.

28. The composition of claim 27, wherein the at least one glycosylated bacterioruberin is present in an amount ranging from 0.0001 to 0.5% by weight with respect to the total weight of the cosmetic composition.

29. A method for preventing oxidative stress in a subject, the method comprising:
administering to the subject a composition comprising at least one glycosylated bacterioruberin, wherein the composition does not comprise a non-glycosylated form of bacterioruberin, and wherein the composition comprises one or more pharmaceutically acceptable excipients.

30. The method of claim 29, wherein the composition:
combats cellular aging of the skin;
scavenges radicals;
protects protein degradation;
protects proteome degradation;
protects cellular detoxification mechanisms; or
protects DNA repair systems.

31. The method of claim 30, wherein the cellular aging of the skin is due to solar radiation, UV radiation, or visible light.

32. The method of claim 29, wherein administering the composition comprises applying the composition to the skin of the subject.
